(19) United States
(12) Patent Application Publication
(10) Pub. No.: US 2026/0226547 A1
(43) Pub. Date: Aug. 6, 2026

(54) GENETIC TESTING FOR EARLY DETECTION OF AUTOIMMUNE DISEASES

(71) Applicant: New York Society for the Relief of the Ruptured and Crippled, maintaining the Hospital for Special S, New York, NY (US)

(72) Inventors: Timothy Niewold, New York, NY (US); Ilona Nln, New York, NY (US)

(73) Assignee: New York Society for the Relief of the Ruptured and Crippled, maintaining the Hospital for Special S, New York, NY (US)

(21) Appl. No.: 19/146,661

(22) PCT Filed: Jan. 11, 2024
(86) PCT No.: PCT/US2024/011178

§ 371 Date: Jul. 9, 2025

Related U.S. Application Data
(60) Provisional application No. 63/438,289, filed on Jan. 11, 2023.

(51) Int. Cl. C12Q 1/6883; G16B 20/20

(52) U.S. Cl. CPC C12Q 1/6883; G16B 20/20; C12Q 2600/156; C12Q 2600/172

(57) ABSTRACT

Methods for detecting an autoimmune disease or a high risk of developing an autoimmune disease in a subject are provided. In a first aspect, a sample from the subject for a haplotype of IRF5 is assayed. The sample is further assayed for a HLA risk allele pair. It is then detected whether the subject has an autoimmune disease or is at high risk for an autoimmune disease when a haplotype of IRF5 and a HLA risk allele pair are present in the sample. The autoimmune disease can be lupus erythematosus (SLE), Sjögren's syndrome (SS), or other autoimmune diseases.

### CROSS-REFERENCE TO RELATED APPLICATIONS

The present application is based on and claims priority to U.S. Provisional Application No. 63/438,289, filed Jan. 11, 2023, and entitled GENETIC TESTING FOR EARLY DETECTION OF AUTOIMMUNE DISEASES, the entire contents of which are hereby incorporated by reference as if expressly set forth in its entirety herein.

### FIELD

The present disclosure relates, generally, to methods for detecting an autoimmune disease, and more particularly to detecting an autoimmune disease or a high risk of developing an autoimmune disease in a subject by assaying a sample from the subject.

### BACKGROUND

Autoimmune diseases, including systemic lupus erythematosus (SLE) and Sjogren's Syndrome (SS), are complex, chronic, and often debilitating illnesses that can go undetected for years.

One of the major reasons for lack of detectability of the diseases is that most patients are asymptomatic until some irreversible tissue damage has occurred, and only then start to develop overt signs and symptoms. Time of detection could make a huge difference in prognosis. If an early diagnosis is made, preventative treatments and close monitoring can be implemented to prevent and reduce the risk of disease progression and avoid organ damage. Although high risk groups, e.g., those with a first degree relative with an autoimmune disease, can be identified, without a definitive early confirmation of disease, the risk of side effects of some therapeutic agents often outweighs the benefits.

Genetic testing for early detection of autoimmune diseases that could be clinically implemented and enable early interventions including participations in clinical trials and/or close monitoring would be of great value clinically.

To date no effective genetic testing is available for autoimmune diseases. There are only a few existing genetic studies disclosing possible at-risk genes, and all of them have limited prediction power.

### SUMMARY

Disclosed herein is a genetic test for the early detection of an autoimmune disease or of a strong risk for an autoimmune disease, with a strong predictive power (an odds ratio (OR) of approximately 300-400), which is greater than 100-fold stronger than the existing tests or published risk factors for autoimmune disease.

The current test is unique not only in the fact that it is highly predictive of disease at an early stage, but because it utilizes a model of multiple interacting genes in complex interactions that result in epistatic effects. Additionally, the test uses a model of multi-dimensional alleles, utilizing multiple loci and strands where each locus and each strand represent one dimension, resulting in a multidimensional risk model.

In one aspect of the disclosure, it has been discovered that there is an increased frequency of an allele of the interferon (IFN) regulatory 5 (IRF5) gene which detects an autoimmune disease and/or identifies a high risk for autoimmune disease (model H1).

Thus, one embodiment of the current disclosure is a method for detecting an autoimmune disease or a high risk of developing an autoimmune disease in a subject, comprising:
a. assaying a sample from the subject for a haplotype of IRF5; and
b. detecting that the subject has an autoimmune disease or is at high risk for developing an autoimmune disease when the haplotype is present in the sample.

In another aspect of the disclosure, a method of detecting an autoimmune disease or a high risk of developing an autoimmune disease in a subject is provided, the method comprising:
a. assaying a sample from the subject for a haplotype of IRF5;
b. further assaying the sample for a HLA risk allele pair; and
c. detecting that the subject has an autoimmune disease or is at high risk for an autoimmune disease when a haplotype of IRF5 and a HLA risk allele pair is present in the sample.

In a further aspect, the subject has a relative with an autoimmune disease.

In some embodiments, the HLA risk allele pair comprises an HLA allele selected from the group consisting of B*801, B*1801, C*701, DQA1*102, DQA1*501, DQB1*201, DQB1*602, DRB1*301, DRB1*1501, DR3, and combinations thereof.

In some embodiments, the HLA risk allele pair comprises an HLA allele selected from the group consisting of A*1, B*8, B*18, C*7, C*12, DQA1*5, DPB1*1, DQB1*2, DQB1*6, DRB1*3, DRB1*15, and combinations thereof.

In some embodiments, the HLA risk allele pair comprises at least DR3.

In some embodiments, the haplotype of IRF5 is selected from the group consisting of TACA, TATA, GCTA, GCTG, and TCTA. In some embodiments, the haplotype of IRF5 is TACA.

In some embodiments, the method further comprises analyzing the HLA risk allele pairs by treating the distinct loci as a different dimension in one or more association analyses.

In some embodiments, the method further comprises a step of computing an odds ratio or a positive predictive value using an algorithm.

In some embodiments, the method further comprises treating the subject if an autoimmune disease or a high risk of autoimmune disease is detected.

In a further aspect of the disclosure, a two-dimensional model was developed using HLA allele pairs based on eight identified HLA risk alleles, which resulted in 21 HLA risk alleles. A 3-class classification system was developed where 11 factors were classified into a high-risk group, 2 factors into a medium-risk group, and 8 into a low-risk group. For each class group, a composite risk factor is defined by the presence of any of the HLA risk allele pairs for each group. For each of low/medium/high risk groups, the presence of at least one HLA risk allele pair equals a risk (HLA+) (model H2). In some embodiments, the risk allele pair comprises at least one HLA allele selected from the group consisting of B*801, B*1801, C*701, DQA1*102, DQA1*501, DQB1*201, DQB1*602, DRB1*301, DRB1*1501, and DR3. In some embodiments, the risk allele pair comprises at least DR3.

Thus, a further embodiment of the current disclosure is a method of detecting an autoimmune disease or a high risk of developing an autoimmune disease in a subject, comprising:
a. assaying a sample from the subject for one of 21 risk allele pairs of HLA; and
b. detecting that the subject has an autoimmune disease or is at high risk for an autoimmune disease when any one of the HLA risk allele pairs is present in the sample.

While either method can be used to detect an autoimmune disease or a high risk of developing an autoimmune disease, the combined method of detecting the risk allele of IRF5 and detecting one of the 21 risk allele pairs of HLA greatly increases the odds ratio in detecting and/or predicting disease, from about 2.0, to about 69.0-225.0 (model H1 and H2).

In a further aspect of the disclosure, the two-dimensional model above (H2) was expanded to N-dimensional, by treating distinct loci as a different dimension in the association analyses. It was found that adding the dimension in the analyses led to a significant enhancement in detecting disease and/or risk for disease. Using the same classification system as for H2, 17 factors were found in the high-risk group and 14 factors in the medium-risk group. For each class group, a composite risk factor is defined by the presence of any of the HLA risk allele pairs for each group. For each of low/medium/high risk groups, the presence of at least one HLA risk allele pair equals a risk (HLA+) (model H3). In some embodiments, the risk allele pair comprises at least one HLA allele selected from the group consisting of B*801, B*1801, C*701, DQA1*102, DQA1*501, DQB1*201, DQB1*602, DRB1*301, DRB1*1501, and DR3. In some embodiments, the risk allele pair comprises at least DR3.

Thus, a further embodiment of the current disclosure is a method of detecting an autoimmune disease or a high risk of developing an autoimmune disease in a subject, comprising:
a. assaying a sample from the subject for one of 31 risk allele pairs of HLA; and
c. detecting that the subject has an autoimmune disease or is at high risk for an autoimmune disease when any one of the 31 risk allele pairs of HLA is present in the sample.

Using this method alone, the odds ratio in detecting and/or predicting disease, is about 104-401.

Using all three methods combined, the odds ratio is cumulative and not additive, as would be expected. The odds ratio in detecting and/or predicting disease when all three methods are used is about 300-400 in most cases, with a very high positive predictive value of about 78% to 99%. The odds ratio of the use of any known risk factor to detect or predict autoimmune disease is about 2.0 in most cases, with positive predictive value of less than 1% in most cases.

Thus, a further embodiment of the current disclosure is a method of detecting an autoimmune disease or a high risk of autoimmune disease in a subject, comprising:
a. assaying a sample from the subject for a haplotype of IRF5;
b. further assaying a sample from the subject for one of 21 risk allele pairs of HLA;
c. further assaying a sample from the subject for one of 31 risk allele pairs of HLA; and
d. detecting that the subject has an autoimmune disease or is at high risk for an autoimmune disease when the risk IRF5 haplotype from step a, one of the 21 risk allele pairs of HLA from step b, and one of the 31 risk allele pairs from step c is present in the sample.

In some embodiments, the haplotype of IRF5 is selected from the group consisting of TACA, TATA, GCTA, GCTG and TCTA. In some embodiments, the haplotype of IRF5 is TACA.

In some embodiments, the risk allele pair comprises at least one HLA allele selected from the group consisting of B*801, B*1801, C*701, DQA1*102, DQA1*501, DQB1*201, DQB1*602, DRB1*301, DRB1*1501, and DR3. In some embodiments, the risk allele pair comprises at least DR3.

Using the models set forth herein, IRF5/HLA risk factor combinations for systemic lupus erythematosus (SLE) and Sjögren's syndrome (SS) were discovered. These risk factor combinations are set forth in Table 1.

Thus, a further embodiment of the current disclosure is a method of detecting an autoimmune disease or a high risk of developing an autoimmune disease in a subject, comprising:
a. assaying a sample from the subject for one of the IRF5/HLA allele combinations listed in Table 1; and
b. detecting that the subject has, or is at high risk for developing an autoimmune disease when IRF5/HLA allele combination is listed in Table 1 as a high risk or medium risk.

In some embodiments, the autoimmune disease is SLE and/or SS.

In some embodiments, the subject has a relative with SLE and/or SS.

The current disclosure also includes a further step or steps for analyzing the detection or presence of the risk alleles to obtain a value, such as an odds ratio, disease prediction score or disease risk score. In some embodiments, the analysis may be performed using an algorithm. In some embodiments, other clinical and/or patient background data is used to obtain the value.

In some embodiments, the method further comprises a step of computing an odds ratio or a positive predictive value using an algorithm.

In some embodiments, the method further comprises treating the subject if an autoimmune disease or a high risk of autoimmune disease is detected.

In a further embodiment of the current disclosure is a method to identify genetic risk factors for autoimmune diseases using allele/haplotype combinations in a multi-dimensional/multi-locus model of HLA and IRF5 genes, comprising:
a. identifying HLA/IRF5 risk factor combinations from HLA allele pairs and IRF5 haplotypes, analyzing the epistatic effects of the HLA/IRF5 combinations, and assigning each analyzed HLA/IRF5 combination to either a high risk group, medium risk group or a low risk group;
b. identifying HLA/IRA5 risk factor combinations from 2-dimensional HLA allele pairs and IRF5 haplotypes, analyzing the epistatic effects of the HLA/IRF5 combinations, and assigning each analyzed HLA/IRF5 combination to either a high risk group, medium risk group or a low risk group;
c. identifying HLA/IRF5 risk factor combinations from 3-dimensional HLA allele pairs and IRF5 haplotype, analyzing the epistatic effects of the HLA/IRF5 combinations, and assigning each analyzed HLA/IRF5 combination to either a high risk group, medium risk group or a low risk group; and
d. combining the identified HLA/IRF5 risk factor combinations in the high risk and medium risk groups from steps a., b. and c. to identify genetic risk factors for autoimmune diseases.

The current disclosure also includes methods of treatment, after an autoimmune disease or risk thereof has been detected.

Autoimmune disease diagnoses that can be detected or predicted using the current methods include but are not limited to systemic lupus erythematosus (SLE), Sjögren's syndrome (SS), rheumatoid arthritis, type 1 diabetes, psoriasis, scleroderma, multiple sclerosis, dermatomyositis, ankylosing spondylitis, celiac disease, Hashimoto's thyroiditis, and myasthenia gravis.

In some embodiments, the autoimmune disease is systemic lupus erythematosus (SLE). In some embodiments, the autoimmune disease is Sjögren's syndrome (SS).

Kits for performing any of the disclosed methods are also provided.

### BRIEF DESCRIPTION OF THE FIGURES

For the purpose of illustrating the invention, there are depicted in drawings certain embodiments of the invention. However, the invention is not limited to the precise arrangements and instrumentalities of the embodiments depicted in the drawings.

FIG. 1 is a flowchart of a clinical implantation of the current disclosure.

FIG. 2—A. Odds ratio and p-value results for Case vs Control outcome, for HLA and IRF5 carriers without HLA-IRF5 interactions. B. Association analysis between HLA+ (allele carrier) and IRF5+(haplotype carrier) gene-gene interaction. The odds ratio bars represent HLA− IRF5−, HLA− IRF5+, HLA+IRF5−, HLA+IRF5+ from left to right respectively. C. Association analysis showing risk epistasis due to HLA+ (DR3 carrier) and IRF5+ (TACA carrier) gene-gene interaction, non-carriers are indicated by the minus postfix (HLA− or IRF5−). The upper and lower 95% confidence intervals are shown by the lines. D. Association analysis results of FIG. 2C. in tabular form.

FIG. 3 shows individual HLA allele pair association analysis in case-control analysis. Results shown for HLA DR3 compounds starting with 135 allele pairs, filtered down to allele pairs that have HLA+IRF5+p-value <0.05 for SLE diagnosis. HLA+ and IRF5+ denote subjects that carry the HLA indicated on the horizontal axis and IRF5 risk haplotypes TACA, TATA, GCTG, GCTA, TCTA indicated on the vertical axis.

FIG. 4 is a graph of the results using the H1+H2 model method, the H3 model method and three model method as compared to the published literature to predict disease in systemic lupus erythematosus (SLE) EA.

FIG. 5 is a graph of the results using the H1+H2 model method, the H3 model method and the three model method to predict disease in systemic lupus erythematosus (SLE) HA.

FIG. 6 is a graph of the results using the H1+H2 model method, the H3 model method and the three model method to predict disease in systemic lupus erythematosus (SLE) AA.

FIG. 7 is a graph of the results using the H1+H2 model method, the H3 model method and the three model method to predict disease in Sjogren's Syndrome (SS) EA.

FIG. 8 is a graph of the results using the H1+H2 model method (model), and variations of the H3 model method (variation of model 2) and the three model H1, H2 and H3 method (variation of model 3) as compared to the published literature to predict disease in systemic lupus erythematosus (SLE) EA.

### DETAILED DESCRIPTION OF THE DISCLOSURE

### Definitions

The terms used in this specification generally have their ordinary meanings in the art, within the context of this invention and the specific context where each term is used. Certain terms are discussed below, or elsewhere in the specification, to provide additional guidance to the practitioner in describing the methods of the invention and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of the other synonyms. The use of examples anywhere in the specification, including examples of any terms discussed herein, is illustrative only, and in no way limits the scope and meaning of the invention or any exemplified term. Likewise, the invention is not limited to its preferred embodiments.

The term “subject” as used in this application means an animal with an immune system such as avians and mammals. Mammals include canines, felines, rodents, bovine, equines, porcines, ovines, and primates. Avians include, but are not limited to, fowls, songbirds, and raptors. Thus, the invention can be used in veterinary medicine, e.g., to treat companion animals, farm animals, laboratory animals in zoological parks, and animals in the wild. The invention is particularly desirable for human medical applications.

The term “patient” as used in this application means a human subject. In some embodiments, the “patient” is one suffering from, diagnosed with, suspected of having and/or considered at risk for an autoimmune disease. In some embodiments, the autoimmune disease is systemic lupus erythematosus. In some embodiments, the autoimmune disease is Sjögren's syndrome. In some embodiments, the patient has not been diagnosed with, suspected of having, or considered at risk for developing an autoimmune disease.

The terms “prediction”, “predict”, “predicting” and the like as used herein means to tell in advance based upon special knowledge.

The terms “treat”, “treatment”, and the like refer to a means to slow down, relieve, ameliorate or alleviate at least one of the symptoms of the disease, or reverse the disease after its onset.

The terms “prevent”, “prevention”, and the like refer to acting prior to overt disease onset, to prevent the disease from developing or minimize the extent of the disease or slow its course of development.

The term “agent” as used herein means a substance that produces or is capable of producing an effect and would include, but is not limited to, chemicals, pharmaceuticals, biologics, small organic molecules, antibodies, nucleic acids, peptides, and proteins.

The phrase “therapeutically effective amount” is used herein to mean an amount sufficient to cause an improvement in a clinically significant condition in the subject, or delays or minimizes or mitigates one or more symptoms associated with the disease, or results in a desired beneficial change of physiology in the subject.

As used herein, the term “isolated” and the like means that the referenced material is free of components found in the natural environment in which the material is normally found. In particular, isolated biological material is free of cellular components. In the case of nucleic acid molecules, an isolated nucleic acid includes a PCR product, an isolated mRNA, a cDNA, an isolated genomic DNA, or a restriction fragment. In another embodiment, an isolated nucleic acid is preferably excised from the chromosome in which it may be found. Isolated nucleic acid molecules can be inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated material may be, but need not be, purified.

The term “purified” and the like as used herein refers to material that has been isolated under conditions that reduce or eliminate unrelated materials, i.e., contaminants. For example, a purified protein is preferably substantially free of other proteins or nucleic acids with which it is associated in a cell; a purified nucleic acid molecule is preferably substantially free of proteins or other unrelated nucleic acid molecules with which it can be found within a cell. As used herein, the term “substantially free” is used operationally, in the context of analytical testing of the material. Preferably, purified material substantially free of contaminants is at least 50% pure; more preferably, at least 90% pure, and more preferably still at least 99% pure. Purity can be evaluated by chromatography, gel electrophoresis, immunoassay, composition analysis, biological assay, and other methods known in the art.

The terms “gene”, “gene transcript”, and “transcript” and “allele” are used somewhat interchangeably in the application. The term “gene”, also called a “structural gene” means a DNA sequence that codes for or corresponds to a particular sequence of amino acids which comprise all or part of one or more proteins or enzymes, and may or may not include regulatory DNA sequences, such as promoter sequences, which determine for example the conditions under which the gene is expressed. Some genes, which are not structural genes, may be transcribed from DNA to RNA, but are not translated into an amino acid sequence. Other genes may function as regulators of structural genes or as regulators of DNA transcription. “Transcript” or “gene transcript” is a sequence of RNA produced by transcription of a particular gene. Thus, the expression of the gene can be measured via the transcript.

“Nucleic acid” refers to deoxyribonucleotides or ribonucleotides and polymers thereof in either single- or double-stranded form. The nucleic acids herein may be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. The nucleic acids may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, and carbamates) and with charged linkages (e.g., phosphorothioates, and phosphorodithioates). Polynucleotides may contain one or more additional covalently linked moieties, such as, for example, proteins (e.g., nucleases, toxins, antibodies, signal peptides, and poly-L-lysine), intercalators (e.g., acridine, and psoralen), chelators (e.g., metals, radioactive metals, iron, and oxidative metals), and alkylators. The polynucleotides may be derivatized by formation of a methyl or ethyl phosphotriester or an alkyl phosphoramidate linkage. Modifications of the ribose-phosphate backbone may be done to facilitate the addition of labels, or to increase the stability and half-life of such molecules in physiological environments. Nucleic acid analogs can find use in the methods of the invention as well as mixtures of naturally occurring nucleic acids and analogs. Furthermore, the polynucleotides herein may also be modified with a label capable of providing a detectable signal, either directly or indirectly. Exemplary labels include radioisotopes, fluorescent molecules, and biotin.

The term “polypeptide” as used herein means a compound of two or more amino acids linked by a peptide bond. “Polypeptide” is used herein interchangeably with the term “protein.”

The term “about” or “approximately” means within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

The current disclosure is a method for the early detection of autoimmune disease and/or detecting a high risk for an autoimmune disease. Early detection is important such that interventions such as treatment and close monitoring can be implemented before disease progression leading to organ damage takes place. Currently there is no effective genetic marker or test that reliably detects the disease or a high risk of disease.

Systemic lupus erythematosus (SLE) is a complex autoimmune disease. Many common alleles with moderate to small effect on SLE risk have been identified, but strong gene-gene interactions have not been reported. Reported herein is an extremely strong SLE risk factor (OR=401, p=2.6×10⁻⁴⁶), composed of an epistatic gene-gene interaction between common haplotypes in the IRF5 locus and combinations of HLA alleles. These combinations were present in 15.9% of European-American SLE patients and none of 1055 European-American controls and were associated with a greater than 2-fold risk of severe clinical manifestation (ACR score>6) within the SLE cohort. Similar IRF5/HLA epistatic interactions were observed in Sjogren's disease and SLE patients of other ancestral backgrounds, supporting relevance of this genetic model across multiple autoimmune diseases and data sets.

The currently disclosed method enables the early detection with a strong odds ratio (OR) of approximately 300-400 and a high positive predictive value of approximately 78% to 99%. Genetic association of this strength (OR>400) has actionable clinical predictive utility in SLE.

This current method uses a multiple interacting gene model that is applicable to genetic testing of other genes in autoimmune diseases. The model utilizes complex interactions of multiple genes that result in epistatic effect. As will be shown, while certain individual IRF5 and HLA haplotypes are associated with autoimmune disease, the odds ratio and frequency of these associations are very low, multitudes less than the current method.

While both IRF5 and HLA are known genetic risk factors for SLE, disclosed herein is the first showing of the two risk alleles interacting to cause autoimmune disease pathogenesis at a significantly higher risk level. Due to the complexity of the interactions, new screening methods and models needed to be developed.

The current method also uses a multi-dimensional allele model (H3), that even used without the H1 and H2 models, has a very strong predictive power. This concept is also applicable to genetic testing of other genes in autoimmune diseases. This model utilizes multiple loci and multiple strands, where each strand and each locus represent one dimension, which results in a multidimensional risk model.

Lastly, the current method uses a composite model that combines multiple risk factors and partitions subjects into defined risk groups (H1, H2 and H3). Using this composite model, IRF5/HLA risk factor combinations for systemic lupus erythematosus (SLE) of European ancestry (EA), Amerindian ancestry (HA) and African ancestry (AA) and Sjögren's syndrome (SS) of European ancestry (EA) were discovered. Examples of these risk factor combinations are set forth in Table 1.

TABLE 1
Risk factor combinations for various diseases and backgrounds

| | | | |
|---|---|---|---|
| SLE EA | SS EA | SLE HA | SLE AA |

High risk group

| | | | |
|---|---|---|---|
| DQB1*201/C*401 & | C*701/C*303 & | A*1101/DPB1*402 & | DRB1*701/DRB1*1302 & |
| TATA | TCTA | TACA | TCTA |
| DR3/B*1501 & | B*801/DQB1*602 & | DQA1*102/DQA1*501 & | DPB1*401/DQB1*201 & |
| TATA | TCTA | TACA | GCTG |
| DR3/DPB1*402 & | DRB1*301/DQB1*604 & | DQA1*501/DQA1*503 & | DPB1*401/DRB1*301 & |
| TACA | TCTA | TACA | GCTG |
| DQA1*102/B*1402 & | A*101/B*3503 & | DQA1*501/DQA1*301 & | DRB1*301/A*3601 & |
| TATA | TATA | TACA | GCTG |
| B*801/DQB1*202 & | DRB1*301/B*1501 & | DQA1*501/A*101 & | DPB1*401/DPB1*602 & |
| TACA | TATA | TCTA | TACA |
| DR3/DQB1*604 & | DRB1*1501/DPB1*501 & | DQA1*102/DPB1*401 & | DPB1*101/ |
| TATA | TATA | TCTA | DQB1*602&DPB1*401 & |
| | | | GCTA |
| B*1801/B*2705 & | B*801/B*3701 & | B*801/DQB1*501 & | DQB1*301/ |
| GCTG | TACA | TATA | DQB1*602&A*6802 & |
| | | | GCTG |
| B*801/B*3801 & | B*801/B*5101 & | C*701/DRB1*1501 & | DPB1*401/ |
| TATA | TACA | TATA | DQA1*102&A*201 & |
| | | | GCTG |
| DRB1*1501/C*1203 & | A*101/B*3501 & | DQA1*102/C*1502 & | |
| TACA | TACA | TATA | |
| B*801/DRB1*1302 & | A*101/DQB1*202 & | DQA1*501/DQA1*401 & | |
| TACA | TACA | TATA | |
| B*1801/C*1203 & | B*801/B*702& | C*701/A*301 & | |
| GCTA | DQB1*301 & TATA | GCTG | |
| B*801/ | DRB1*1501/ | A*1101/DQA1*102 & | |
| DPB1*401&B*801 & | DRB1*301&DPB1*201 & | GCTG | |
| TATA | TATA | | |
| DPB1*401/ | B*801/C*701& | A*1101/DQB1*201 & | |
| DQA1*501&DQB1*501 & | B*3701 & TACA | GCTG | |
| TACA | | | |
| DQA1*501/B*702& | A*201/A*101& | DRB1*1501/DPB1*402 & | |
| DPB1*201 & TATA | C*501 & TACA | GCTG | |
| DRB1*1501/ | A*101/DQA1*501& | B*801/DQB1*501 & | |
| DPB1*401&DQA1*101 & | B*3501 & TACA | GCTG | |
| TACA | | | |
| C*701/DPB1*401& | DRB1*301/ | C*701/A*3201 & | |
| DQB1*501 & TACA | DPB1*401&A*3201 & | GCTG | |
| | TACA | | |
| DPB1*401/C*701& | | C*701/DQA1*401 & | |
| DQB1*602 & TACA | | GCTG | |
| DPB1*401/ | | DQA1*102/A*201 & | |
| DQA1*102&C*304 & | | GCTA | |
| TACA | | | |
| DQA1*501/ | | DQA1*102/B*1801 & | |
| DQA1*102&C*401 & | | GCTA | |
| TACA | | | |
| DQB1*201/C*701& | | DQA1*102/DPB1*402 & | |
| DQB1*301 & TACA | | GCTA | |
| DQB1*602/ | | DRB1*1501/ | |
| DR3&C*304 & | | DRB1*301&A*101 & | |
| TATA | | GCTA | |
| B*801/ | | DQB1*602/ | |
| B*801&DPB1*101 & | | DQA1*102&A*201 & | |
| GCTG | | GCTA | |
| DRB1*1501/ | | DPB1*401/ | |
| DQA1*102&DPB1*201 & | | DQB1*602&DPB1*401 & | |
| TACA | | TACA | |
| B*801/ | | DRB1*1501/ | |
| C*701&DQB1*604 & | | DQA1*102&C*701 & | |
| TATA | | TATA | |
| B*801/DQA1*501& | | A*1101/DPB1*402 & | |
| C*1203 & TACA | | TACA | |
| DQA1*101/ | | | |
| DQB1*602&DPB1*402 & | | | |
| TACA | | | |
| DR3/ | | | |
| DQA1*501&B*5101 & | | | |
| TACA | | | |
| DQB1*201/ | | | |
| DQA1*501&B*1801 & | | | |
| TACA | | | |

Medium risk group

| | | | |
|---|---|---|---|
| DRB1*1501/B*1801 & | A*101/C*1203 & | DQA1*102/DPB1*401 & | DRB1*701/DRB1*1503 & |
| GCTA | TATA | GCTA | TCTA |
| DQA1*501/C*102 & | B*801/B*3503 & | DQB1*602/DPB1*401 & | DPB1*401/A*2301& |
| TATA | TATA | GCTA | GCTG |
| DQB1*201/ | B*801/DQB1*202 & | DRB1*1501/DPB1*401 & | B*801/B*5301 & |
| DQA1*501&B*3801 & | TCTA | GCTA | GCTG |
| TATA | | | |
| DQB1*602/ | B*801/B*4402 & | DQB1*602/A*201 & | DQB1*602/A*201 & |
| DPB1*401&B*801 & | TCTA | GCTA | GCTG |
| TACA | | | |
| DR3/ | A*101/A*3201 & | DQA1*501/C*701 & | DRB1*301/DRB1*1503 & |
| DQA1*501&B*1501 & | TCTA | TACA | GCTG |
| TATA | | | |
| DQA1*501/ | DRB1*301/DRB1*1501 & | B*801/DPB1*401 & | DRB1*301/DQB1*602 & |
| DQA1*102&DPB1*201 & | TACA | TACA | GCTG |
| TATA | | | |
| DQA1*102/ | DRB1*301/A*2902 & | DRB1*301/DRB1*1501 & | DQB1*602/DQA1*101 & |
| DQA1*501&DQB1*604 & | TACA | TACA | GCTG |
| TATA | | | |
| DQA1*102/ | DQB1*602/DQB1*604 & | | DRB1*301/DQB1*602 & |
| DQA1*501&DPB1*402 & | TACA | | GCTA |
| TACA | | | |
| DRB1*1501/ | | | DPB1*401/DQA1*102 & |
| DPB1*401&DQB1*501 & | | | GCTA |
| TACA | | | |
| DQA1*102/B*1402 | | | DRB1*301/B*5301 & |
| &DPB1*401 & | | | TATA |
| GCTG | | | |
| C*701/DQA1*501& | | | |
| DQB1*301 & TACA | | | |
| DQA1*101/ | | | |
| DRB1*1501&DPB1*402 & | | | |
| TACA | | | |
| DR3/C*701&DQB1*301 & | | | |
| TACA | | | |
| DQA1*102/ | | | |
| DQA1*501&C*802 & | | | |
| TATA | | | |
| B*801/DQA1*501& | | | |
| B*1801 & TACA | | | |
| DQA1*102/ | | | |
| DQA1*501&B*4001 & | | | |
| TATA | | | |
| DRB1*1501/B*1801 & | | | |
| GCTA | | | |
| DQA1*501/C*102 & | | | |
| TATA | | | |
| DQB1*201/ | | | |
| DQA1*501&B*3801 & | | | |
| TATA | | | |
| DQB1*602/ | | | |
| DPB1*401&B*801 & | | | |
| TACA | | | |
| DR3/ | | | |
| DQA1*501&B*1501 & | | | |
| TATA | | | |
| DQA1*501/ | | | |
| DQA1*102&DPB1*201 & | | | |
| TATA | | | |

Low risk group

DQA1*501/
DQA1*501 & TACA
DQA1*501/
DRB1*101 & TACA
DQA1*102/C*501 &
TACA
B*801/DQB1*301 &
TACA
DR3/DRB1*1302 &
TACA
B*1801/DPB1*101 &
TACA
DR3/DRB1*1302 &
TATA
B*1801/DPB1*101 &
TATA

Protective group

| | | | |
|---|---|---|---|
| DQA1*102/DQA1*103 & | C*701/DQA1*202 & | DQA1*102/DRB1*1302 & | DPB1*101/DQB1*602 & |
| TATA | TATA | TACA-neg | GCTG |
| DQA1*102/DRB1*1601 & | | DQA1*501/DQB1*301 & | |
| TATA | | TACA-neg | |
| DR3/DQA1*505 & | | | |
| TATA | | | |
| B*801/DPB1*101 & | | | |
| GCTA | | | |

The method of the current disclosure utilizes at least one model of analysis of risk alleles. In further embodiments, the method utilizes more than one model of analysis of risk alleles. In some embodiments, the risk alleles analyzed are IRF5 alleles. In some embodiments, the risk alleles analyzed are HLA alleles. In some embodiments, risk alleles from both HLA and IRF5 are analyzed. i.e., an IRF5/HLA genotype is analyzed.

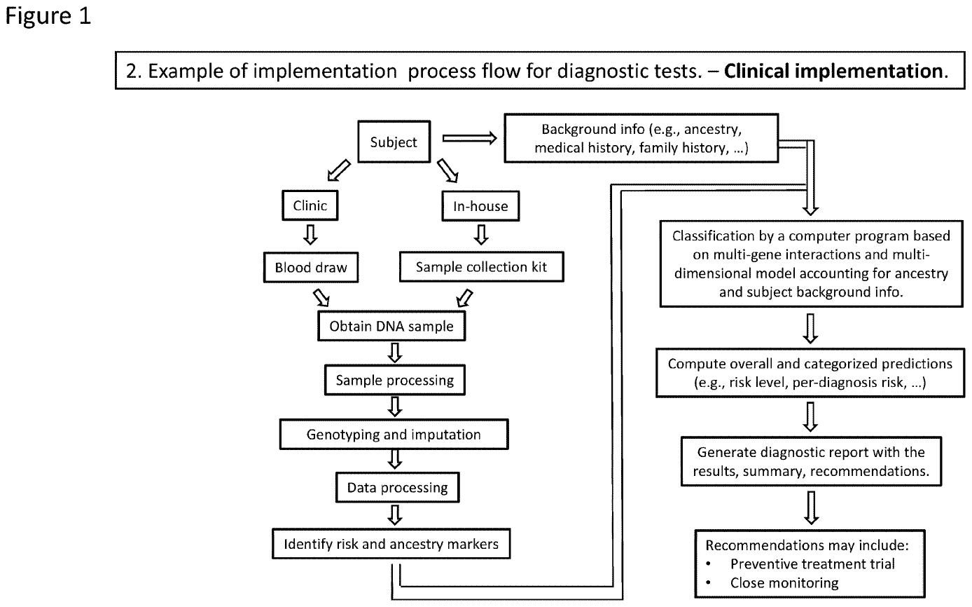

Moreover, the currently disclosed method can be clinically implemented. See FIG. 1.

The strength of this gene-gene interaction upon SLE risk produces an actionable result. SLE has a prevalence of 164 per 100,000 (0.16%) in European ancestry women. Using combined high-risk group frequency and Monte Carlo simulation, the frequencies of high-risk group for each cohort in healthy population was estimated. Then the positive predictive values for each cohort as a measure of clinical diagnostic value was computed. In SLE European ancestry the high-risk group has a PPV of 77.77% quantifying the probability for individuals that carry the high-risk factors to develop SLE. For Amerindian ancestry SLE the PPV is 86.7%, African American ancestry SLE PPV is 91.4%, and European ancestry Sjogren's PPV is 82.3%.

While screening the general population may not be feasible, those in a high-risk group such as those with first-degree relatives of SLE patients (Sinicato et al. 2019; Kuo et al. 2015), could be screened using the methods disclosed herein. A high-risk individual that carries the risk IRF5/HLA genotype would have an almost determinative probability of developing the disease. This strong probability of disease is clinically meaningful for early detection of autoimmune diseases and can provide actionable options for at-risk subjects to seek preventive treatment or close monitoring program. The risk IRF5/HLA genotype disclosed herein can be useful for recruitment for clinical trials as well.

Individually, the HLA and IRF5 genes are known to be associated with SLE with a mild odds ratio of approximately 2 (Graham et al. (2007a), Niewold et al. (2012), Langefeld et al. (2017)). The novel model developed and disclosed herein accounts for epistatic interactions between the HLA and IRF5 genes and demonstrates significantly stronger association with SLE with an odds ratio of approximately 178. The model is further enhanced by another novel multi-dimensional model that enables an even stronger association with SLE with an odds ratio of approximately 401, or approximately 100-fold stronger as compared to the published literature. These findings open up a path to clinical genetic testing with significant detection power that can be very valuable for individuals especially those who are in a high-risk group such as first-degree relatives of an SLE subject.

Interferon (IFN) regulatory 5 (IRF5) is encoded by the human IRF5 gene located at chromosome 7q32 (OMIM ID 607218). IRF5 is a member of the IRF family; it is a transcription factor that possesses a helix-turn-helix DNA-binding motif and mediates virus- and interferon (IFN)-induced signaling pathways. It is appreciated that several isoforms/transcriptional variants of IRF5 are known. It is also well known that IRF5 is polymorphic, and a large number of polymorphisms, including SNPs are known. While IRF5 haplotypes have been associated with SLE, the odds ratio or frequency in all of those published reports was very low. See, e.g., Niewold et al. (2008); Niewold et al. (2012); Graham et al. (2007); Zervou et al. (2017).

HLAs corresponding to MHC class I (A, B, and C) which all are the HLA Class I group present peptides from inside the cell. In general, these particular peptides are small polymers, about 9 amino acids in length. Foreign antigens presented by MHC class I attract killer T-cells (also called CD8 positive- or cytotoxic T-cells) that destroy cells. MHC class I proteins associate with β2-microglobulin, which unlike the HLA proteins is encoded by a gene on chromosome 15.

HLAs corresponding to MHC class II (DP, DM, DO, DQ, and DR) present antigens from outside of the cell to T-lymphocytes. These particular antigens stimulate the multiplication of T-helper cells (also called CD4 positive T cells), which in turn stimulate antibody-producing B-cells to produce antibodies to that specific antigen. Self-antigens are suppressed by regulatory T cells. The affected genes are known to encode 4 distinct regulatory factors controlling transcription of MHC class II genes.

HLAs corresponding to MHC class III encode components of the complement system.

Aside from the genes encoding the 6 major antigen-presenting proteins, there are a large number of other genes, many involved in immune function, located on the HLA complex.

Each human cell expresses six MHC class I alleles (one HLA-A, -B, and -C allele from each parent) and six to eight MHC class II alleles (one HLA-DP and -DQ, and one or two HLA-DR from each parent, and combinations of these). The MHC variation in the human population is high, at least 350 alleles for HLA-A genes, 620 alleles for HLA-B, 400 alleles for DR, and 90 alleles for DQ. In humans, MHC class II molecules are encoded by three different loci, HLA-DR, -DQ, and -DP, which display about 70% similarity to each other. Polymorphism is a notable feature of MHC class II genes. The HLA region has been strongly associated with autoantibody profiles in SLE patients, including the DR3 allele with anti-Ro antibodies (Graham et al. 2007a). This association makes sense biologically, as the HLA region encodes the MHC molecules which present antigens to the immune system. Thus, it is logical that variations in these molecules could result in abnormal self-antigen presentation and risk of developing an inappropriate immune response against self-peptides.

The current methods include assaying for or detecting certain alleles in a sample from a subject. The disclosed methods can be carried out in numerous ways, by a diagnostic laboratory, and/or a health care provider.

In some embodiments, a haplotype of IRF5 is detected. In some embodiments, the haplotype TACA of IRF5 is detected.

In some embodiments, a HLA risk allele pair is detected. In some embodiments, the risk allele pair comprises at least B*801, B*1801, C*701, DQA1*102, DQA1*501, DQB1*201, DQB1*602, DRB1*301, DRB1*1501, or DR3. In some embodiments, the risk allele pair comprises DR3.

In some embodiments, both a haplotype of IRF5 and an HLA risk allele pair is detected. In some embodiments, the haplotype TACA of IRF5 is detected. In some embodiments, the risk allele pair comprises at least B*801, B*1801, C*701, DQA1*102, DQA1*501, DQB1*201, DQB1*602, DRB1*301, DRB1*1501, or DR3. In some embodiments, the risk allele pair comprises DR3.

In some embodiments, a haplotype of IRF5 and an HLA risk allele pair listed in Table 1 is detected.

In some embodiments, the subject has not been diagnosed with, suspected of having, or considered at risk for developing an autoimmune disease. In some embodiments, the subject has been diagnosed with having an autoimmune disease. In some embodiments, the is suspected of having an autoimmune disease. In some embodiments, the subject is considered at risk for developing an autoimmune disease. In some embodiments, the subject has a first degree relative with an autoimmune disease.

Autoimmune disease diagnoses that can be detected or predicted using the current methods include but are not limited to systemic lupus erythematosus (SLE), Sjögren's syndrome, rheumatoid arthritis, type 1 diabetes, psoriasis, scleroderma, multiple sclerosis, dermatomyositis, ankylosing spondylitis, celiac disease, Hashimoto's thyroiditis, and myasthenia gravis.

In some embodiments, the autoimmune disease is systemic lupus erythematosus (SLE). In some embodiments, the disease is Sjogren's Syndrome.

The sample from the subject includes but is not limited to bone marrow, whole blood, plasma, saliva, and urine.

The nucleic acid is extracted, isolated and purified from the cells of the tissue or fluid by methods known in the art.

If required, a nucleic acid sample is prepared using known techniques. For example, the sample can be treated to lyse the cells, using known lysis buffers, sonication, electroporation, with purification and amplification occurring as needed, as will be understood by those in the skilled in the art. In addition, the reactions can be accomplished in a variety of ways. Components of the reaction may be added simultaneously, or sequentially, in any order. In addition, the reaction can include a variety of other reagents which can be useful in the methods and assays and would include but is not limited to salts, buffers, neutral proteins, such albumin, and detergents, which may be used to facilitate optimal hybridization and detection, and/or reduce non-specific or background interactions. Also reagents that otherwise improve the efficiency of the assay, such as protease inhibitors, nuclease inhibitors, and anti-microbial agents, can be used, depending on the sample preparation methods and purity.

Methods for detecting alleles are often hybridization based and include Southern blots; Northern blots; dot blots; primer extension; nuclease protection; subtractive hybridization and isolation of non-duplexed molecules using, for example, hydroxyapatite; solution hybridization; filter hybridization; amplification techniques such as RT-PCR and other PCR-related; fingerprinting, such as with restriction endonucleases; and the use of structure specific endonucleases.

One method for the detection of the alleles is the use of arrays or microarrays. These terms are used interchangeably and refer to any ordered arrangement on a surface or substrate of different molecules, referred to herein as “probes.” Each different probe of any array is capable of specifically recognizing and/or binding to a particular molecule, which is referred to herein as its “target” in the context of arrays. Examples of typical target molecules that can be detected using microarrays include mRNA transcripts, cRNA molecules, cDNA, PCR products, and proteins.

Microarrays are useful for simultaneously detecting the presence, absence and quantity of a plurality of different target molecules in a sample. The presence and quantity, or absence, of the probe's target molecule in a sample may be readily determined by analyzing whether and how much of a target has bound to a probe at a particular location on the surface or substrate.

In a preferred embodiment, arrays used in the present invention are “addressable arrays” where each different probe is associated with a particular “address.”

Any additional method known in the art can be used to detect the presence or absence of the alleles.

For a general description of these techniques, see also Sambrook et al. 1989; Kriegler 1990; and Ausebel et al. 1990.

Screening and diagnostic method of the current invention may involve the amplification of the target loci. A preferred method for target amplification of nucleic acid sequences is using polymerases, in particular polymerase chain reaction (PCR). PCR or other polymerase-driven amplification methods obtain millions of copies of the relevant nucleic acid sequences which then can be used as substrates for probes or sequenced or used in other assays.

Amplification using polymerase chain reaction is particularly useful in the embodiments of the current invention. PCR is a rapid and versatile in vitro method for amplifying defined target DNA sequences present within a source of DNA. Usually, the method is designed to permit selective amplification of a specific target DNA sequence(s) within a heterogeneous collection of DNA sequences (e.g. total genomic DNA or a complex cDNA population). To permit such selective amplification, some prior DNA sequence information from the target sequences is required. This information is used to design two oligonucleotide primers (amplimers) which are specific for the target sequence and which are often about 15-25 nucleotides long.

In some embodiments, after sample processing, i.e., IRF5/HLA risk allele detection, data processing and classification are performed to compute a value, such as an odds ratio, disease prediction score or disease risk score. See FIG. 1. In some embodiments, this value is computed using an algorithm. In some embodiments, other clinical data and/or patient background variables are used in computing the value. In some embodiments, a computer configured by code executing therein is used to perform the algorithm. In some embodiments, a system is used which can comprise a processor, configured by code executing therein, to obtain a plurality of measurement values for the patient. The processor of the system is configured by code executing therein, to provide the plurality of measurement values as inputs to a predictive model. In one particular configuration, the predictive model is configured to output a value, such as an odds ratio, disease prediction score or disease risk score, in response to the input values. In some embodiments, the system will also include a means for communicating with a database. In some embodiments, the system will also include a means for obtaining the measurement values from the patient.

The current disclosure also includes methods of treatment, after an autoimmune disease or risk thereof has been detected.

In the case of SLE, such treatments can include anti-malarial agents, corticosteroids, and disease-modifying anti-rheumatic drugs (DMARDS) are immunomodulatory agents that act as immunosuppressives and cytotoxic and anti-inflammatory medications.

Anti-malarial agents work with subtle immunomodulation without causing overt immunosuppression. These drugs are useful in preventing and treating lupus skin rashes, constitutional symptoms, arthralgias, and arthritis. Anti-malarials also help to prevent lupus flares and have been associated with reduced morbidity and mortality in SLE patients followed in observational trials. Anti-malarial drugs include hydroxychloroquine.

Corticosteroid agents are used predominantly for anti-inflammatory activity and as immunosuppressants. Preparations include oral, intravenous, topical, and intra-articular injections. Corticosteroids include methylprednisolone, which is used for acute organ-threatening exacerbations.

Prednisone is the most common immunosuppressant for treatment of autoimmune disorders and is the steroid most commonly prescribed for lupus. Low-dose oral prednisone can be used for milder SLE, but more severe involvement necessitates high doses of oral or intravenous therapy.

Prednisone is usually given as tablets that come in 1, 5, 10, or 20 milligram (mg) doses. Pills may be taken as often as 4 times a day or as infrequently as once every other day. Usually, a low dose of prednisone is less than 20 mg/day, a medium dose is between 7.5 and 30 mg per day, and a dose of more than 30 mg qualifies as a high dose.

Disease-modifying anti-rheumatic drugs (DMARDS) are immunomodulatory agents that act as immunosuppressives and cytotoxic and anti-inflammatory medications. The specific agent selection is generally indicated by the patient's organ involvement and disease severity. Due to toxicity, cyclophosphamide is reserved for severe organ-threatening disease. At the other end of the spectrum, methotrexate or azathioprine may be helpful for milder arthritis or skin disease. DMARDS can be used in patients whose condition has had an inadequate response to glucocorticoids.

Cyclophosphamide is used for immunosuppression in cases of serious SLE organ involvement, especially severe CNS involvement, vasculitis, and lupus nephritis.

Methotrexate is used for managing arthritis, serositis, cutaneous, and constitutional symptoms. It blocks purine synthesis and 5-aminoimidazole-4-carboxamide ribonucleotide (AICAR), thus increasing anti-inflammatory adenosine concentration at sites of inflammation. Methotrexate ameliorates symptoms of inflammation and is particularly useful in arthritis treatment.

Azathioprine is an immunosuppressant and a less toxic alternative to cyclophosphamide. It is used as a steroid-sparing agent in nonrenal disease.

Mycophenolate is useful for maintenance in lupus nephritis and other serious lupus cases. This agent inhibits inosine monophosphate dehydrogenase (IMPDH) and suppresses de novo purine synthesis by lymphocytes, thereby inhibiting their proliferation. Mycophenolate also inhibits antibody production.

Additionally, patients diagnosed with SLE or any other autoimmune disease can be monitored carefully by a clinician or health care provider at least quarterly.

In the case of Sjogren's Syndrome (SS), such treatments can include, for example, medications for decreasing eye inflammation, such as immunosuppressive eye drops and/or eye drops to treat dry eyes (e.g., cyclosporine, lifitegrast); medications that can increase product of saliva and/or tears (e.g., pilocarpine, cevimeline); nonsteroidal anti-inflammatory drugs (NSAIDs); arthritis medications; antimalaria treatments (e.g., hydroxychloroquine); and immunosuppressants (e.g., methotrexate).

It is contemplated that all of the methods and/or assays disclosed herein can be in kit form for use by a health care provider and/or a diagnostic laboratory.

### EXAMPLES

The present invention may be better understood by reference to the following non-limiting examples, which are presented in order to more fully illustrate the preferred embodiments of the invention. They should in no way be construed to limit the broad scope of the invention.

### Example 1—Materials and Methods

### Patients and Samples

SLE patients were enrolled in the registry having met the American College of Rheumatology classification criteria for SLE (Tan et al. 1982) and Sjogren's patients met the American-European Consensus Group (AECG) criteria. Patients were recruited at the Oklahoma Medical Research Foundation (648 European-American, 234 Amerindian, and 366 African-American SLE patient samples, and 264 European-American Sjogren's patient samples). Healthy controls included 552 European-American, 203 Amerindian, and 252 African-American control samples from OMRF. Subject data from the 1000 Genomes database was also examined as an additional control data set (503 European-American, 347 Amerindian, and 661 African-American control samples). Genetic ancestry was confirmed in all subjects (Langefeld et al. 2017). The study was approved by the institutional review boards at the institution noted.

### Genotyping

Subjects in the OMRF registry and the healthy controls were genotyped and passed quality control for the Immunochip genotyping platform. HLA haplotypes were imputed from the SNPs available on the chip, and the IRF5 risk alleles were directly typed on this platform (rs2004640, rs3807306, rs10488631, and rs2280714). For PCR genotyping, all plots were inspected visually for quality control. IRF5 alleles were imputed using Michigan Imputation Server and phased using Eagle v2.4.1 (Loh et al. (2016)). Haplotype frequencies in SLE patients and controls were similar to published data (Graham et al. (2007)). HLA alleles were imputed for all subjects using the same Michigan Imputation Server and HIBAG (Loh et al. (2016)). HLA alleles with a posterior probability >0.9 were taken forward into subsequent analyses.

### Autoantibody Measurement

In the OMRF samples, autoantibodies were measured using the Ouchterlony method (Clark et al. (1969)). Autoantibody data was coded as positive or negative based upon the recommended cutoff used in the respective clinical laboratories and was used in the analysis as a categorical variable.

### Example 2—Model Development

It has been previously shown that a haplotype of IRF5 is associated with anti-Ro antibodies in both SLE patients and healthy individuals (Niewold et al. 2012; Cherian et al. 2012). Interestingly, IRF5 risk allele carriage was also a strong risk factor for progression from the asymptomatic anti-Ro positive state to a clinical diagnosis of SLE (OR=5.8, p=2.4×10⁻³) (Cherian et al. (2012)). The HLA region has been strongly associated with autoantibody profiles in SLE patients, including the DR3 allele with anti-Ro antibodies (Graham et al. (2007); Hamilton et al. (1988)). Therefore, it was hypothesized that IRF5 variants may interact with SLE-associated HLA variants to predispose to autoimmunity.

In developing model H1, a logistic regression model was not used. The model involved a single IRF5 feature (single strand carriers), with no weighting coefficients. An example is TACA/XXXX or XXXX/TACA (any strand carrier). IRF5 risk carrier is defined by single-strand presence of specific IRF5 haplotype. If a specific haplotype is present equals Risk carrier (IRF5+); if a specific haplotype is absent equals Non risk carrier (IRF5−).

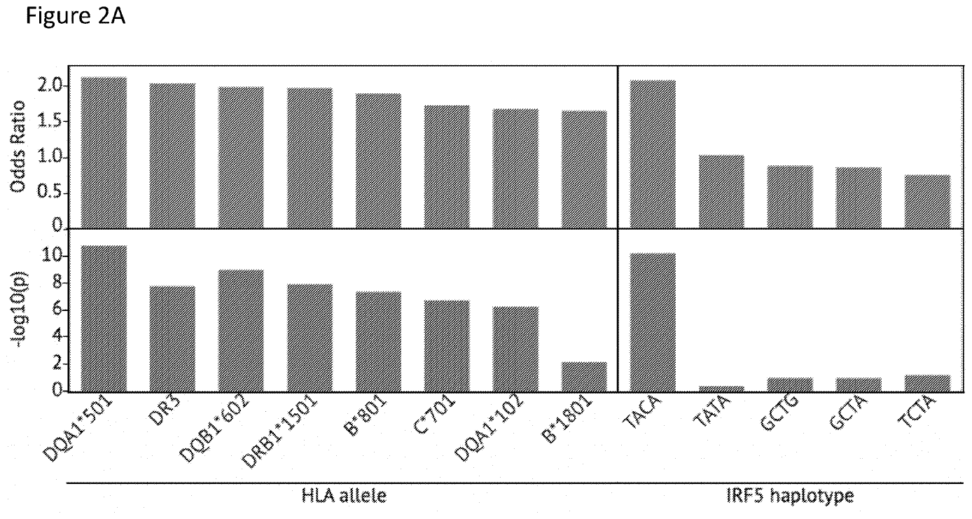

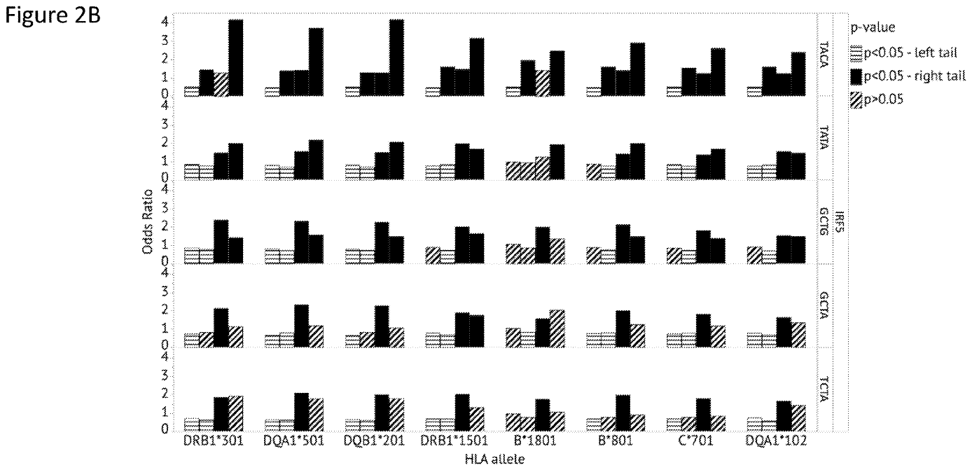

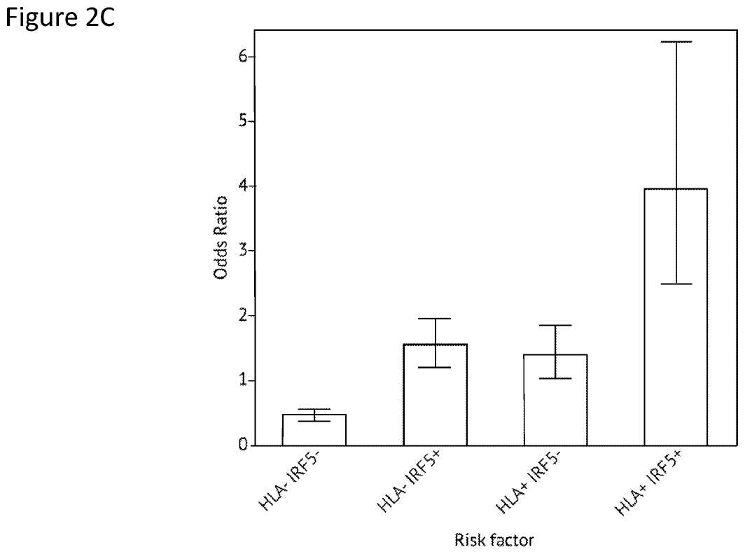

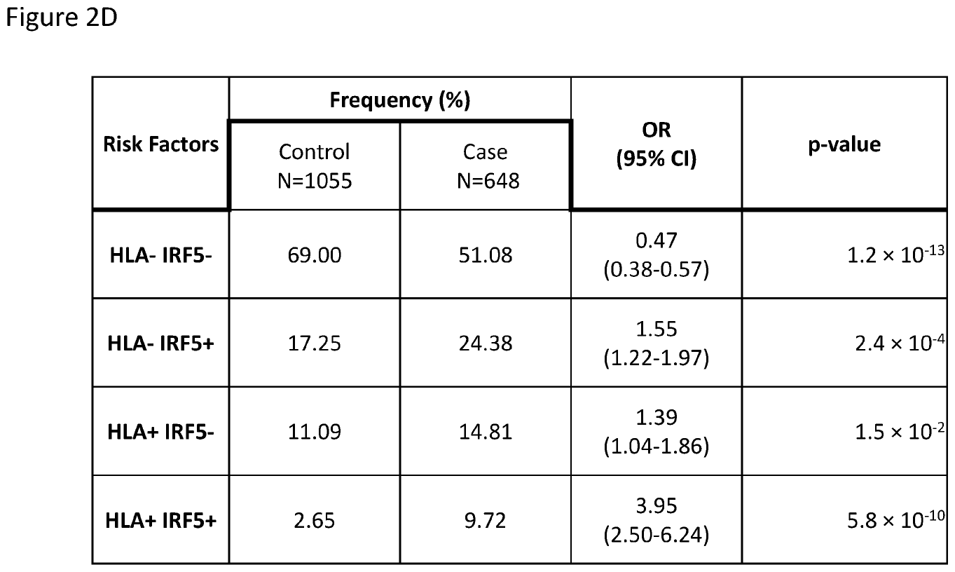

The IRF5 common haplotype TACA (rs2004640, rs3807306, rs10488631, rs2280714) has been associated with SLE (Graham et al. (2007a)). This is in agreement with the result that shows OR=2.08 (1.67-2.6) with p=6.5 e−11 for TACA as a significant risk factor for SLE (FIG. 2A).

In developing model H2 with HLA alleles, association analyses were performed for 135 alleles on 7 HLA loci (A, B, C, DPB1, DQA1, DQB1 and DRB1), which resulted in 8 significant HLA risk factors (DQA1*501; DR3; DQB1*602; DRB1*1501; B*801; C*701; DQA1*102; B*1801) that have OR>1.5 and p<0.05 (FIG. 2A).

To investigate the hypothesized HLA-IRF5 epistatic interaction (model H1 and H2), association analyses was performed for all combinations of 8 HLA risk factors and 5 IRF5 common haplotypes (TACA, TATA, GCTG, GCTA, TCTA), where 4 cases are computed for each HLA-IRF5 combination (−−, −+, +−, ++) where +/− denotes presence/absence of the risk factor (FIG. 2B). Multiple epistatic interactions were identified, the strongest of which is for DR3 HLA and TACA IRF5 risk factors (FIGS. 2C, 2D). Here, clear epistatic interaction effects were seen where the lower 95% confidence interval for HLA+IRF5+odds ratio was higher than the upper 95% confidence interval for HLA+IRF5−and HLA-IRF5+.

A number of general observations can be made from FIG. 2B: All of the IRF5 TACA risk factors showed OR++>Max (OR+−, OR−+); IRF5 TATA haplotype was neutral where OR++− OR+− for HLA risk carriers, and was protective for HLA risk non-carriers, OR−+<1; The rest of the IRF5 common haplotypes (GCTG, GCTA, TCTA) overall showed protective effects where OR−+<1 and OR++<OR+−.

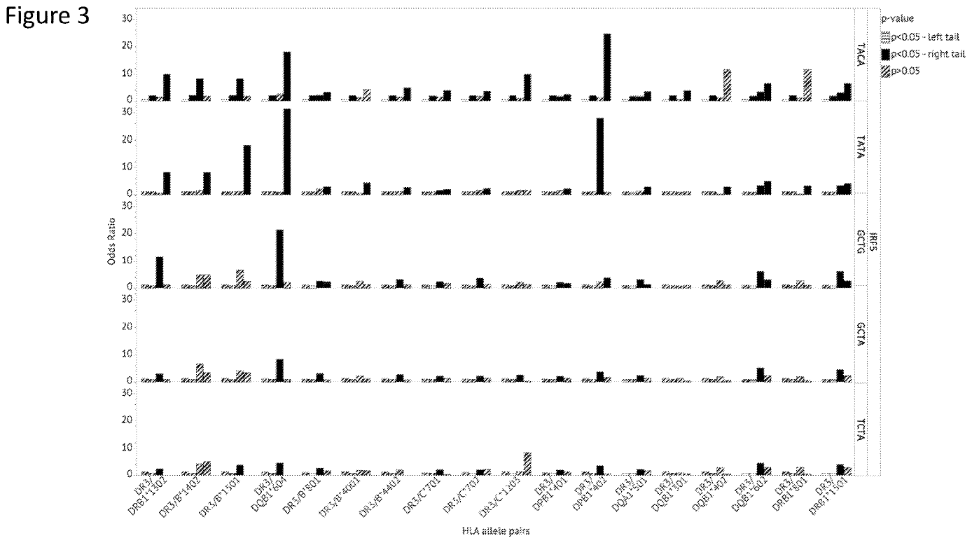

To further investigate HLA-IRF5 epistatic interactions at a finer resolution, association analysis was performed for the 8 identified HLA risk alleles (FIG. 2A) paired with individual HLA alleles in 7 loci, labeled as H1/H2 respectively, and with the 5 IRF5 common haplotypes. There were 1080 HLA H1/H2 pairs for each IRF5 haplotype, for each of the 4 cases (−−, −+, +−, ++). In total, this corresponded to 21,600 HLA-IRF5 combinations, where 286 were associated as risk for SLE (p<0.05) with odds ratios ranging from 1.36-31.36 and 4 were protective for SLE (p<0.05) with odds ratios ranging from 0.08-0.3. At this granular HLA pairs level, significant SLE risk factors with epistatic enhancement of the odds ratios (OR++>Max (OR−+, OR+−)) were identified across all IRF5 common haplotypes, where there are 109 HLA-TACA, 79 HLA-TATA, 33 HLA-GCTG, 11 HLA-GCTA, and 1 HLA-TCTA risk factors. The results for H1=DR3 are shown in FIG. 3, for European-American ancestry subjects, filtered to combinations that have p++<0.05 for at least one IRF5 haplotype. Overall, at this resolution, the HLA DR3 pairs (DR3/H2) showed the strongest SLE risk associations, with significant epistatic effect (FIG. 3). For example, for DR3/DPB1*402 and TACA, when none or only one of the risk factors are present, the odds ratio is around 1, but when the two risk factors are present together, the odds ratio increased to 24.7 (1.4-432.9) and p=1.1 e−3. Further, while DR3/DPB1*402 show strong epistatic risk enhancement for TACA, a clear absence of epistasis interaction was observed for TATA, GCTA, TCTA, and an additive interaction was observed for GCTG. GCTG was previously shown as protective haplotypes, here an increased risk of odds ratio to SLE was observed when GCTG interacted with certain HLA allele pairs. In addition, some of these specific gene-gene interactions showed a protective effect which could also be useful clinically. These results highlight the gene-gene specificity involved in the interaction.

Backward regression was then applied that resulted in 21 HLA significant risk factors and defined a 3-class classification system where 11 factors are classified into a high-risk group, 2 factors into a medium-risk group and 8 into a low-risk group.

To develop model H3, H2 was expanded from 2-D to N-dimensional, by treating distinct loci as a different dimension in the association analyses. Expanding on the dimensionality and specificity of the HLA model into multiple allele combinations across multiple loci on either chromosome, potentially capturing more specific sub-interactions effects, significantly enhanced the model strength. For example, for DQB1*201/C*701 & TACA combination, an addition of DQB1*301 on the same chromosome as C*701 increased the odds ratio for case vs control by about 8-fold. In this expanded model, ~71,000 HLA-IRF5 combinations were analyzed, with HLA pairs having H1/H2 & H3 and H1 & H3/H2 combinations, for specific IRF5 haplotypes, and across the 4 cases (−−, −+, +−, ++). This resulted in ~500 specific combinations with >95% statistically significant association with risk for SLE (p<0.05) with odds ratios ranging from 1.62 to 38.1.

Using the same regression method and classification system as used above, 17 factors were found to be in a high-risk group and 14 factors in a medium-risk group in the expanded H3 model. A composite risk factor model was defined by the presence of any of the HLA risk allele pairs for each group. The HLA-IRF5 composite risk factor was found in 15.9% of 648 SLE patients and was not found in any of the 552 control subjects nor 503 subjects in the European ancestry 1000 Genomes public data set.

The frequency of the risk combination observed in controls is consistent with the expected frequency given known allele frequencies and the assumption of independent assortment of these loci that reside on different chromosomes (calculated at ~1 in 14,000 people).

Clinical associations between the risk genotype combination in SLE were analyzed as compared to non-risk genotype SLE patients. Interestingly, the risk of more severe clinical manifestations was significantly higher in high-risk genotype carriers' group, OR=2.1(1.1-3.83), p=1.8 e−2.

Given that SLE is one of the most common autoimmune diseases associated with SS, it was hypothesized that a similar model may apply to Sjogren's susceptibility. A difference in association between IRF5 haplotypes and Sjogren's vs. SLE was previously observed (Cherian et al. (2012)), and based upon this IRF5 haplotypes that contain rs2004640 T allele carriage (TACA, TATA, TCTA) were used as the risk designation for IRF5. Using a similar algorithm searching for compound HLA interactions with IRF5, a model with a similar strong epistatic association was found between IRF5 and HLA. This composite risk factor was present in 12.5% of Sjogren's patients and 0 out of 1055 controls. The list of risk factors can be found in Table 1. There is one HLA-IRF5 risk factor combination that is shared between SLE and SS, and two other HLA alleles that are shared with SLE but pair with different IRF5 alleles in SS. This suggests that there could be similar mechanisms or pathways shared between SLE and Sjogren's.

A similar model was applied to non-European ancestry SLE patients that indicates similar epistatic effects and risk associations. In Amerindian ancestry SLE, evidence was found for a similar epistatic interaction with specific sets of risk factors. In African-American subjects, an epistatic relationship between HLA and IRF5 with specific sets of risk factors again was observed. There is one HLA risk factor shared between Amerindian and African American but with different IRF5 combinations.

### Example 3—Clinical Results

The various models were applied to patient registries in Example 1, including patients with SLE of European ancestry (EA), Amerindian ancestry (HA), African ancestry (AA), and Sjogren's syndrome EA. In each case, three methods were used: combination of the H1 and H2 model (model 1); the H3 model (model 2); and the combination of all three, H1, H2 and H3 (model 3).

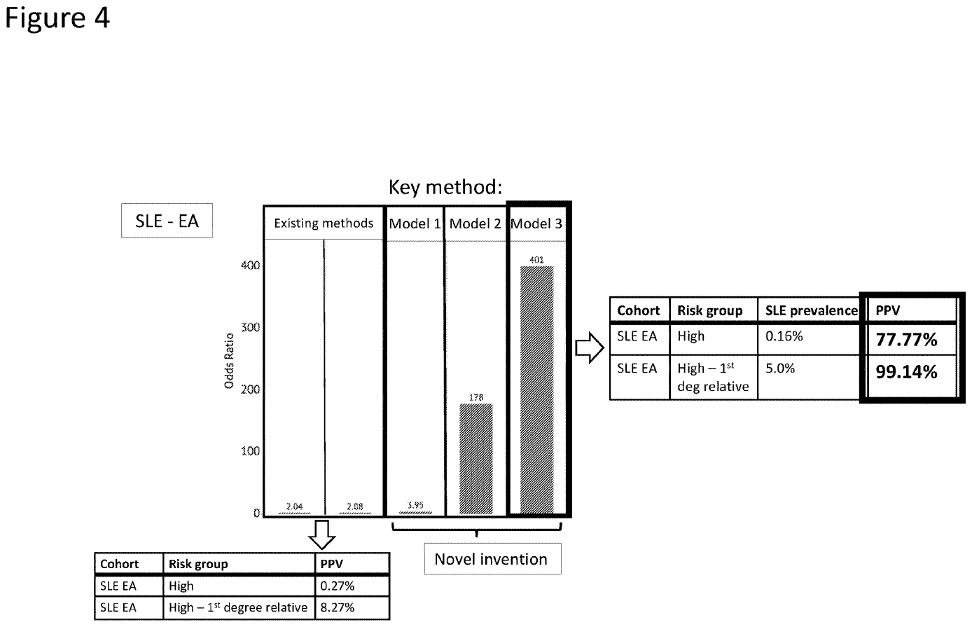

As shown in FIG. 4, when applied to SLE-EA patients, the odds ratio of model 1 was 3.95, model 2, 178, and model 3, 401. In comparison, models developed in the published literature have odds ratios of 1.3-2-7 (Graham et al. (2007a); Niewold et al. (2012); Langefeld et al. (2017)). The odds ratio represents the odds of a subject to develop SLE if tested positive, relative to a negative test.

Additionally, the positive predictive value (which is defined as the probability that the disease is present when the test is positive) of model 3 is 99.14% for SLE patients at high risk with a first degree relative, and 77.77% for SLE patients at high risk, as compared to 8.27% and 0.27% using the existing methods. See Table 2. The positive predictive value was computed using Model 3 and an MC simulation model for control frequency estimation. As exemplified by the results in FIG. 4 and Table 2, the full model (e.g., all 3 models) gives a very strong prediction power. It is also applicable for other diagnoses as well (e.g., Sjogren's syndrome: OR~300).

TABLE 2
PPV estimation using various models in SLE-EA

| | | SLE | | |
|---|---|---|---|---|
| Cohort | Risk Group | prevalence | Model | PPV |
| SLE EA | High | 0.16% | Known | 0.27% |
| SLE EA | High | 0.16% | Model 3 (H1 + | 77.77% |
| | | | H2 + H3+) | |
| SLE EA | High - 1^{st} | 0.16% | Known | 8.27% |
| | degree | | | |
| | relative | | | |
| SLE EA | High - 1^{st} | 0.16% | Model 3 (H1 + | 99.14% |
| | degree | | H2 + H3+) | |
| | relative | | | |

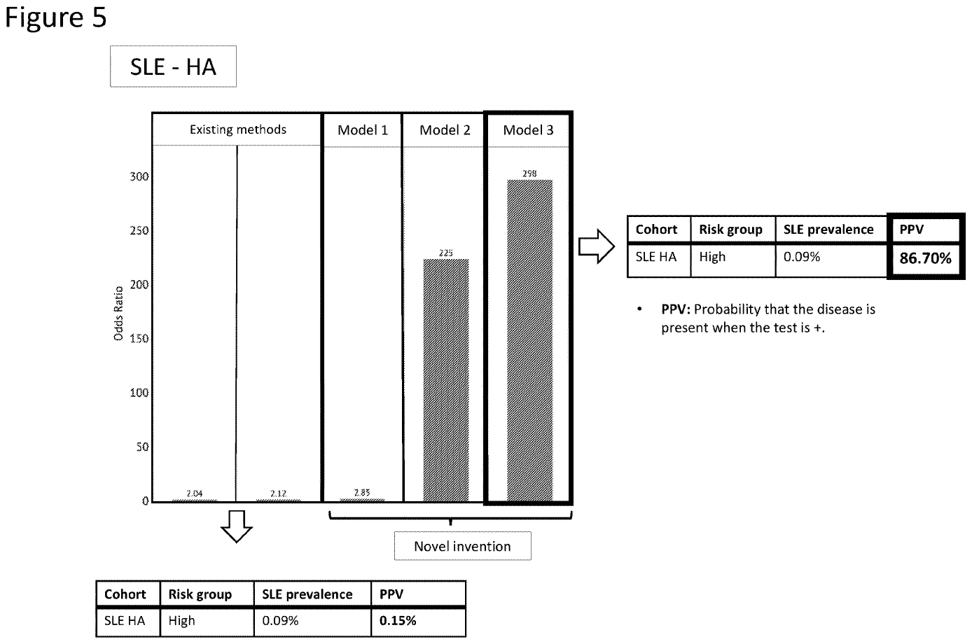

As shown in FIG. 5, when applied to SLE-HA patients, the odds ratio of model 1 was 2.85, model 2, 225, and model 3, 298. In comparison, models developed in the published literature have odds ratios of ~2 (Langefeld et al. (2017); Alarcón-Riquelme et al. (2016)).

Additionally, the positive predictive value of model 3 is 86.70% for SLE patients at high risk, as compared to 0.15% using the existing methods. See Table 3.

TABLE 3
PPV estimation using various models in SLE-HA

| | | SLE | | |
|---|---|---|---|---|
| Cohort | Risk Group | prevalence | Model | PPV |
| SLE HA | High | 0.09% | Known | 0.15% |
| SLE HA | High | 0.09% | Model 3 (H1 + | 86.70% |
| | | | H2 + H3+) | |

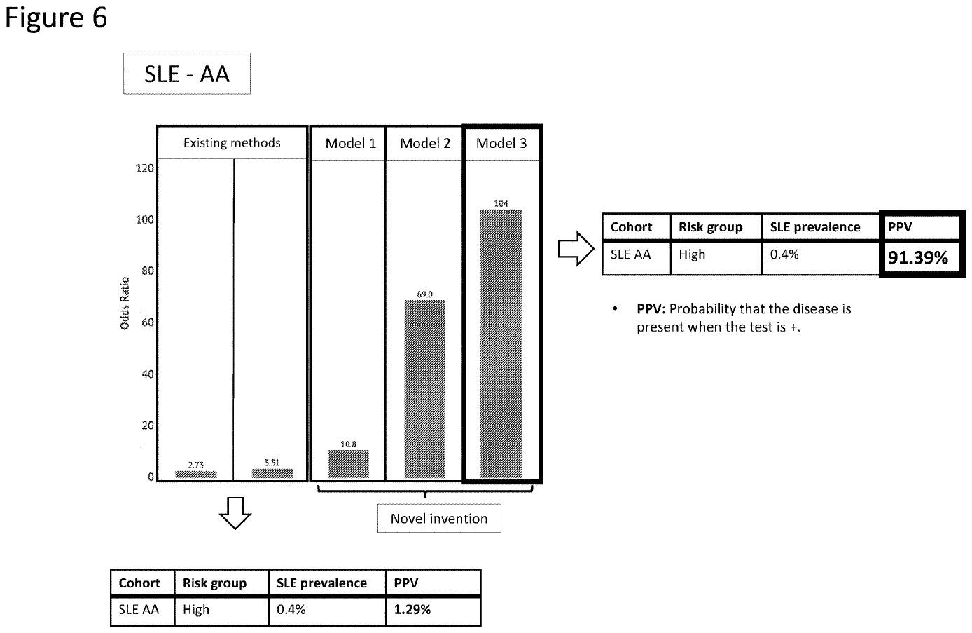

As shown in FIG. 6, when applied to SLE-AA patients, the odds ratio of model 1 was 10.8, model 2, 69, and model 3, 104. In comparison, models developed in the published literature have odds ratios of ~2 (Niewold et al. (2012); Langefeld et al. (2017)).

Additionally, the positive predictive value of model 3 is 91.39% for SLE patients at high risk, as compared to 1.29% using the existing methods. See Table 4.

TABLE 4
PPV estimation using various models in SLE-AA

| | | SLE | | |
|---|---|---|---|---|
| Cohort | Risk Group | prevalence | Model | PPV |
| SLE AA | High | 0.4% | Known | 1.29% |
| SLE AA | High | 0.4% | Model 3 (H1 + | 91.39% |
| | | | H2 + H3+) | |

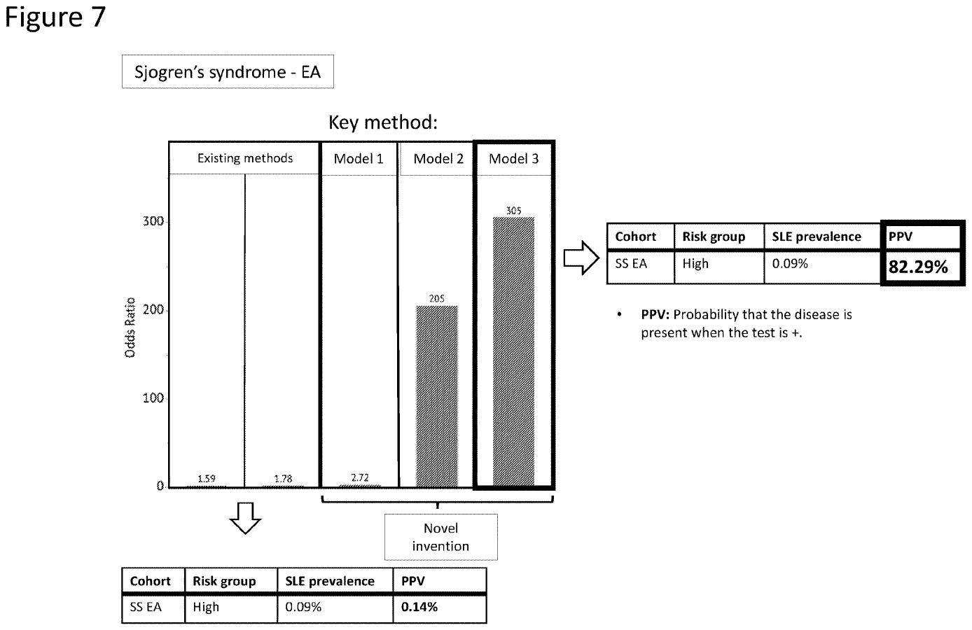

As shown in FIG. 7, when applied to Sjogren's syndrome (SS)-EA patients, the odds ratio of model 1 was 2.72, model 2, 205, and model 3, 305. In comparison, models developed in the published literature have odds ratios of ~2 (Lessard et al. (2012); Taylor et al. (2017)).

Additionally, the positive predictive value of model 3 is 82.29% for SS patients at high risk, as compared to 0.14% using the existing methods. See Table 5.

TABLE 5
PPV estimation using various models in SS-EA

| | | SS | | |
|---|---|---|---|---|
| Cohort | Risk Group | prevalence | Model | PPV |
| SS EA | High | 0.09% | Known | 0.14% |
| SS EA | High | 0.09% | Model 3 (H1 + | 82.29% |
| | | | H2 + H3+) | |

Thus, it can be seen from the clinical result, that while all disclosed models are superior in detecting early disease or risk of disease, the method utilizing H1+H2+H3 is vastly superior in its ability to detect and predict disease in all SLE irrespective of origin and SS.

### Example 4—Additional Clinical Results

Similar to Example 3, in this example various models were applied to patient registries in Example 1, specifically patients with SLE of European ancestry (EA). Of note, in this example a variant of the model 2 and model 3 described above were used. The variants of models 2 and 3 use 2-digit coding of the HLA alleles, as compared with the 4-digit coding using in the original models 2 and 3 described above.

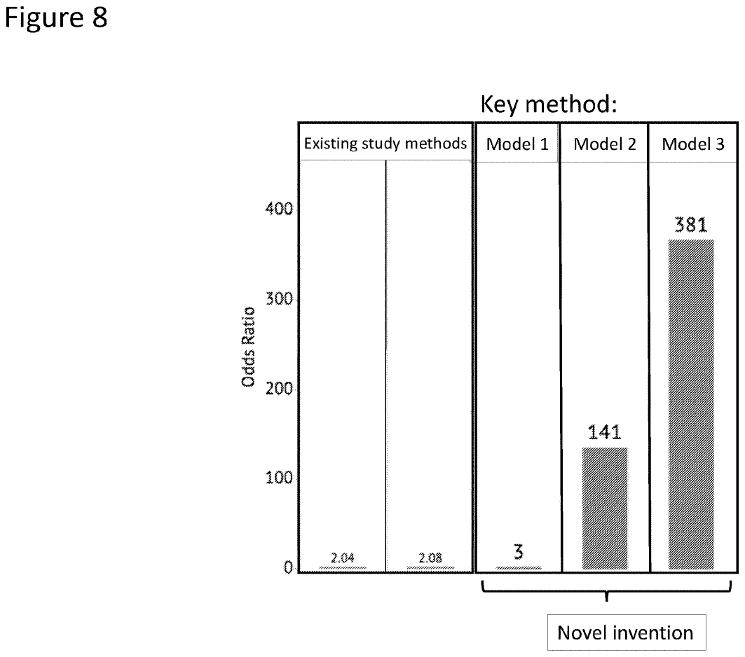

As shown in FIG. 8, when applied to SLE-EA patients, the odds ratio of model 1 was 3, variant model 2, 141, and variant model 3, 381. In comparison, models developed in the published literature have odds ratios of 1.3-2-7 (Graham et al. (2007a); Niewold et al. (2012); Langefeld et al. (2017)). The odds ratio represents the odds of a subject to develop SLE if tested positive, relative to a negative test.

Table 6 below shows the results for the risk HLA allele pairs for this example. Specifically, using the composite model of model 1 and variant models 2 and 3, examples of IRF5/HLA risk factor combinations for systemic lupus erythematosus (SLE) European ancestry (EA) were discovered, as set forth in Table 6.

The results of Example 4 indicate even greater improvement in SLE diagnosis (for EA) using the composite model of model 1 and the variants of models 2 and 3 that utilize 2-digit coding for of the HLA alleles as compared with the models that utilize 4-digit coding.

TABLE 6
risk factor combinations for SLE EA
SLE EA

High risk group
DRB1*3/DRB1*8 & TACA
C*12/DQA1*1 & TACAhom
DPB1*1/C*14 & GCTG
DRB1*15/A*11 & GCTA
A*1/DRB1*15 & TACAhom
DQB1*2/DPB1*15 & GCTA
DRB1*3/DPB1*2 & GCTA
DRB1*3/DPB1*4 & TACAhom
B*18/B*27 & GCTG
B*18/B*27 & TATA
DRB1*15/DRB1*7&A*2 & TCTA
DRB1*15/C*5&B*44 & TACA
A*1/DRB1*4&C*7 & TATAhom
C*7/B*49&C*7 & TATAhom
DPB1*1/B*35&C*7 & GCTA
B*13/A*1&C*6 & GCTG
DRB1*16/C*7&DQB1*5 & GCTG
DPB1*1/B*27&C*7 & TATA
DRB1*4/DRB1*3&DPB1*4 & TACA
DRB1*13/DQA1*5&DRB1*13 & TACA
B*7/C*7&A*3 & TACAhom
A*30/B*8&DRB1*3 & TATA
B*27/A*1&DPB1*4 & TATA
B*37/DRB1*3&C*6 & TATA
B*44/DRB1*3&A*1 & TATAhom
B*51/B*8&DQA1*1 & GCTGhom
C*7/B*51&DQA1*5 & GCTGhom
A*11/DRB1*3&A*1 & TACA
Medium risk group
DRB1*15/DRB1*1 & TACA
C*12/DRB1*1 & GCTA
C*12/DRB1*1 & TACA
DQB1*2/A*23&B*35 & TACA
B*51/B*8&A*1 & GCTGhom
A*1/B*18 & TACA
B*8/A*33 & TATA
C*7/B*58 & TATA
DPB1*1/B*40 & TACA
Low risk group
B*8/B*7 & GCTA
DQB1*2/DRB1*15 & TCTA
DRB1*15/DQB1*5 & TACA
DPB1*1/B*8&DPB1*4 & TACA
DRB1*3/B*18&C*7 & TATA
DRB1*4/DRB1*3&A*1 & TACA
Protective group
C*7/A*24 & GCTA
C*7/C*12 & GCTGhom
C*12/A*2 & GCTGhom
DPB1*1/B*44 & TATAhom
DQA1*5/A*29 & GCTG
DQA1*5/DRB1*4 & TATA
DRB1*15/DRB1*15 & GCTGhom

In accordance with one or more embodiments of the present application, and methods are set out in the following items:

Item 1. A method of detecting an autoimmune disease or a high risk of developing an autoimmune disease in a subject, comprising:
a. assaying a sample from the subject for a haplotype of IRF5;
b. further assaying the sample for a HLA risk allele pair; and
c. detecting that the subject has an autoimmune disease or is at high risk for an autoimmune disease when a haplotype of IRF5 and a HLA risk allele pair is present in the sample.

Item 2. The method of item 1, wherein the autoimmune disease is selected from the group consisting of systemic lupus erythematosus (SLE), Sjögren's syndrome (SS), rheumatoid arthritis, type 1 diabetes, psoriasis, scleroderma, multiple sclerosis, dermatomyositis, ankylosing spondylitis, celiac disease, Hashimoto's thyroiditis, and myasthenia gravis.

Item 3. The method of item 1 or item 2, wherein the autoimmune disease is selected from the group consisting of systemic lupus erythematosus (SLE) and Sjögren's syndrome (SS).

Item 4. The method of any one of items 1-3, wherein the subject has a relative with an autoimmune disease.

Item 5. The method of any one of items 1-4, wherein the haplotype of IRF5 is selected from the group consisting of TACA, TATA, GCTA, GCTG and TCTA.

Item 6. The method of any one of items 1-5, wherein the haplotype of IRF5 is TACA.

Item 7. The method of any one of items 1-6, wherein the HLA risk allele pair comprises an HLA allele selected from the group consisting of B*801, B*1801, C*701, DQA1*102, DQA1*501, DQB1*201, DQB1*602, DRB1*301, DRB1*1501, DR3, and combinations thereof.

Item 8. The method of any one of items 1-6, wherein the HLA risk allele pair comprises an HLA allele selected from the group consisting of A*1, B*8, B*18, C*7, C*12, DQA1*5, DPB1*1, DQB1*2, DQB1*6, DRB1*3, DRB1*15, and combinations thereof.

Item 9. The method of any one of items 1-8, wherein the HLA risk allele pair comprises at least DR3.

Item 10. The method of any one of items 1-9, further comprising analyzing the HLA risk allele pairs by treating the distinct loci as a different dimension in one or more association analyses.

Item 11. The method of any one of items 1-10, further comprising a step of computing an odds ratio or a positive predictive value using an algorithm.

Item 12. The method of any one of items 1-11, further comprising treating the subject if an autoimmune disease or a high risk of autoimmune disease is detected.

Item 13. A method of detecting systemic lupus erythematosus (SLE) and/or Sjögren's syndrome (SS) or a high risk of developing SLE and/or SS in a subject, comprising:
a. assaying a sample from the subject for one of the IRF5/HLA allele combinations listed in Table 1 or listed in Table 6; and
b. detecting that the subject has SLE and SS or is at high risk of SLE or SS when the IRF5/HLA allele combination in the sample is listed for a high risk or medium risk group for SLE and/or SS.

Item 14. The method of item 13, wherein the subject has a relative with SLE and/or SS.

Item 15. The method of item 13 or 14, further comprising a step of computing an odds ratio or a positive predictive value using an algorithm.

Item 16. The method of any one of items 13-15, further comprising treating the subject if an autoimmune disease or a high risk of autoimmune disease is detected.

Item 17. A method to identify genetic risk factors for autoimmune diseases using allele/haplotype combinations in a multi-dimensional/multi-locus model of HLA and IRF5 genes, comprising:
a. identifying HLA/IRF5 risk factor combinations from HLA allele pairs and IRF5 haplotypes, analyzing the epistatic effects of the HLA/IRF5 combinations, and assigning each analyzed HLA/IRF5 combination to either a high risk group, medium risk group or a low risk group;
b. identifying HLA/IRA5 risk factor combinations from 2-dimensional HLA allele pairs and IRF5 haplotypes, analyzing the epistatic effects of the HLA/IRF5 combinations, and assigning each analyzed HLA/IRF5 combination to either a high risk group, medium risk group or a low risk group;
c. identifying HLA/IRF5 risk factor combinations from 3-dimensional HLA allele pairs and IRF5 haplotype, analyzing the epistatic effects of the HLA/IRF5 combinations, and assigning each analyzed HLA/IRF5 combination to either a high risk group, medium risk group or a low risk group; and
d. combining the identified HLA/IRF5 risk factor combinations in the high risk and medium risk groups from steps a., b. and c. to identify genetic risk factors for autoimmune diseases.

### REFERENCES

Alarcón-Riquelme et al. Genome-Wide Association Study in an Amerindian Ancestry Population Reveals Novel Systemic Lupus Erythematosus Risk Loci and the Role of European Admixture. Arthritis & rheumatology (Hoboken.) vol. 68,4 (2016)
Cherian et al., Brief Report: IRF5 systemic lupus erythematosus risk haplotype is associated with asymptomatic serologic autoimmunity and progression to clinical autoimmunity in mothers of children with neonatal lupus. Arthritis Rheum-Us 64, 3383-3387 (2012).
Clark et al., Characterization of a soluble cytoplasmic antigen reactive with sera from patients with systemic lupus erythamatosus. J Immunol 102, 117-122 (1969).
Graham et al., Three functional variants of IFN regulatory factor 5 (IRF5) define risk and protective haplotypes for human lupus. Proc. Natl. Acad. Sci. USA 104, 6758-6763 (2007a).
Graham et al., Specific combinations of HLA-DR2 and DR3 class II haplotypes contribute graded risk for disease susceptibility and autoantibodies in human SLE. Eur. J. Hum. Genet. 15, 823-830 (2007).
Hamilton et al., Two Ro (SS-A) autoantibody responses in systemic lupus erythematosus. Correlation of HLA-DR/DQ specificities with quantitative expression of Ro (SS-A) autoantibody. Arthritis Rheum 31, 496-505 (1988).
Kuo et al., Familial Aggregation of Systemic Lupus Erythematosus and Coaggregation of Autoimmune Diseases in Affected Families. JAMA Intern. Med. 175, 1518-1526 (2015)
Langefeld et al., Transancestral mapping and genetic load in systemic lupus erythematosus. Nat Commun 8, 16021 (2017).
Lessard et al. Variants at multiple loci implicated in both innate and adaptive immune responses are associated with Sjögren's syndrome. Nat Genet, 45:1284 (2013)
Loh et al., Reference-based phasing using the Haplotype Reference Consortium panel. Nat Genet 48, 1443-1448 (2016).
Niewold et al., IRF5 haplotypes demonstrate diverse serological associations which predict serum interferon alpha activity and explain the majority of the genetic association with systemic lupus erythematosus. Annals of the rheumatic diseases 71, 463-468 (2012).
Niewold et al., Association of the IRF5 haplotype with high serum interferon-alpha activity in systemic lupus erythematosus patients. Arthritis Rheum. 58, 2481-87 (2008)
Sinicato et al., Familial aggregation of childhood and adulthood-onset Systemic Lupus Erythematosus. Arthritis Care Res. (Hoboken), (2019).
Tan et al., The 1982 revised criteria for the classification of systemic lupus erythematosus. Arthritis Rheum 25, 1271-1277 (1982).
Taylor et al. Genome-Wide Association Analysis Reveals Genetic Heterogeneity of Sjögren's Syndrome According to Ancestry. Arthritis & rheumatology (Hoboken) vol. 69,6 (2017)
Zervou et al., Association of IRF5 polymorphisms with increased risk of systemic lupus erythematosus in population of Crete, a southern-eastern Greek island. Gene 610, 9-14 (2017).

While operations shown and described herein may be in a particular order, this should not be understood as requiring that such operations be performed in the particular order shown or in sequential order, or that all illustrated operations be performed, to achieve desirable results. In certain circumstances, multitasking and parallel processing can be advantageous. Moreover, the separation of various system components in the embodiments described above should not be understood as requiring such separation in all embodiments, and it should be understood that the described program components and systems can generally be integrated together in a single software product or packaged into multiple software products.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, when used in this disclosure, specify the presence of stated features, integers, steps, operations, elements, and/or components, but do not preclude the presence or addition of one or more other features, integers, steps, operations, elements, components, and/or groups thereof.

It should be noted that use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Particular embodiments of the subject matter described in this disclosure have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing can be advantageous.

What is claimed:

1. A method of detecting an autoimmune disease or a high risk of developing an autoimmune disease in a subject, comprising:
a. assaying a sample from the subject for a haplotype of IRF5;
b. further assaying the sample for a HLA risk allele pair; and
c. detecting that the subject has an autoimmune disease or is at high risk for an autoimmune disease when a haplotype of IRF5 and a HLA risk allele pair is present in the sample.

2. The method of claim 1, wherein the autoimmune disease is selected from the group consisting of systemic lupus erythematosus (SLE), Sjögren's syndrome (SS), rheumatoid arthritis, type 1 diabetes, psoriasis, scleroderma, multiple sclerosis, dermatomyositis, ankylosing spondylitis, celiac disease, Hashimoto's thyroiditis, and myasthenia gravis.

3. The method of claim 1, wherein the autoimmune disease is selected from the group consisting of systemic lupus erythematosus (SLE) and Sjögren's syndrome (SS).

4. The method of claim 2, wherein the subject has a relative with an autoimmune disease.

5. The method of claim 1, wherein the haplotype of IRF5 is selected from the group consisting of TACA, TATA, GCTA, GCTG and TCTA.

6. The method of claim 5, wherein the haplotype of IRF5 is TACA.

7. The method of claim 1, wherein the HLA risk allele pair comprises an HLA allele selected from the group consisting of B*801, B*1801, C*701, DQA1*102, DQA1*501, DQB1*201, DQB1*602, DRB1*301, DRB1*1501, DR3, and combinations thereof.

8. The method of claim 1, wherein the HLA risk allele pair comprises an HLA allele selected from the group consisting of A*1, B*8, B*18, C*7, C*12, DQA1*5, DPB1*1, DQB1*2, DQB1*6, DRB1*3, DRB1*15, and combinations thereof.

9. The method of claim 7, wherein the HLA risk allele pair comprises at least DR3.

10. The method of claim 8, wherein the HLA risk allele pair comprises at least DR3.

11. The method of claim 1, further comprising analyzing the HLA risk allele pairs by treating the distinct loci as a different dimension in one or more association analyses.

12. The method of claim 1, further comprising a step of computing an odds ratio or a positive predictive value using an algorithm.

13. The method of claim 1, further comprising treating the subject if an autoimmune disease or a high risk of autoimmune disease is detected.

14. A method of detecting systemic lupus erythematosus (SLE) and/or Sjögren's syndrome (SS) or a high risk of developing SLE and/or SS in a subject, comprising:
a. assaying a sample from the subject for one of the IRF5/HLA allele combinations listed in Table 1 or listed in Table 6; and
b. detecting that the subject has SLE and SS or is at high risk of SLE or SS when an IRF5/HLA allele combination in the sample is listed for a high risk or medium risk group for SLE and/or SS.

15. The method of claim 14, wherein the subject has a relative with SLE and/or SS.

16. The method of claim 14, further comprising a step of computing an odds ratio or a positive predictive value using an algorithm.

17. The method of claim 14, further comprising treating the subject if an autoimmune disease or a high risk of autoimmune disease is detected.

18. A method to identify genetic risk factors for autoimmune diseases using allele/haplotype combinations in a multi-dimensional/multi-locus model of HLA and IRF5 genes, comprising:
a. identifying HLA/IRF5 risk factor combinations from HLA allele pairs and IRF5 haplotypes, analyzing the epistatic effects of the HLA/IRF5 combinations, and assigning each analyzed HLA/IRF5 combination to either a high risk group, medium risk group or a low risk group;
b. identifying HLA/IRA5 risk factor combinations from 2-dimensional HLA allele pairs and IRF5 haplotypes, analyzing the epistatic effects of the HLA/IRF5 combinations, and assigning each analyzed HLA/IRF5 combination to either a high risk group, medium risk group or a low risk group;
c. identifying HLA/IRF5 risk factor combinations from 3-dimensional HLA allele pairs and IRF5 haplotype, analyzing the epistatic effects of the HLA/IRF5 combinations, and assigning each analyzed HLA/IRF5 combination to either a high risk group, medium risk group or a low risk group; and
d. combining the identified HLA/IRF5 risk factor combinations in the high risk and medium risk groups from steps a., b., and c. to identify genetic risk factors for autoimmune diseases.
